(19) United States
(12) Patent Application Publication
(10) Pub. No.: US 2023/0010845 A1
(43) Pub. Date: Jan. 12, 2023

(54) SMART GARMENT HAVING A CONTACT ELECTRODE

(71) Applicant: Adaptive Regelsysteme Gesellschaft M.B.H., Salzburg (AT)

(72) Inventor: Silke Wohnsdorf, Salzburg (AT)

(21) Appl. No.: 17/782,405

(22) PCT Filed: Dec. 2, 2020
(86) PCT No.: PCT/EP2020/084193

§ 371 Date: Jun. 3, 2022

(30) Foreign Application Priority Data
Dec. 4, 2019 (AT) A51058/2019

(51) Int. Cl. A41D 1/00; H01B 5/14

(52) U.S. Cl. CPC A41D 1/005; H01B 5/14

(57) ABSTRACT

Various embodiments of the present disclosure are directed to electrode arrangements. In one example electrode arrangement, the arrangement includes an electrically conductive electrode, an electrically conductive thermoadhesive, and an electrode carrier. The electrode carrier is provided in the form of a flat material piece consisting of a textile or non-textile material. The electrode carrier is connected on one side to the electrically conductive electrode and the electrically conductive thermoadhesive being in contact with an opposite side of the electrode carrier. The electrode and the thermoadhesive are electrically conductively connected to form an electrical connection

The present invention relates to a smart clothing having a carrier material on which an electrode is arranged, and to a method for producing a smart clothing comprising an electrode, and to an electrode arrangement for forming the electrode.

Electrodes, in particular body electrodes, are required, for example, to measure electrical voltages on a human or animal body in order to measure muscle activity or the pulse. However, electrodes are also required for other technical applications, such as the detection of current flows through the human body in order to be able to detect electrical accidents. However, electrodes are also required to introduce currents into the human or animal body, as is necessary for electrical muscle stimulation (EMS) or for resuscitation measures.

However, electrodes are also required for contacting other items, in particular for contacting electrically conductive items, such as metallic items. A conceivable application is, for example, in the technical inspection of welding points, in which the electrical resistance of the welding point is to be determined using four-wire measurement. In order to prevent visual impairment to the item, four electrodes, two on each side of the item, can be pressed onto the item without impairing the item, as would otherwise be the case, for example, with hard clamps.

In addition, there is of course a large number of other applications in which an electrode is required for contacting an item.

In particular in the case of human or animal applications, but also in other applications, electrodes made of skin-friendly materials such as silicone or polytetrafluoroethylene (PTFE) or other plastics materials can be used.

A material for the electrode that ensures a low contact resistance is preferred in order to allow good electrical contacting. From this point of view, electrically conductive silicone or PTFE, or generally a plastics material having good electrical conductivity, is a good selection for the material of the electrode.

Electrodes are also increasingly being used in clothing to form what are known as smart clothing, also referred to as smart textiles. Clothing comprising integrated electronics or electrical systems that are sometimes also not visible from the outside are referred to as smart textiles. Almost any functions can be achieved using the electronics, such as monitoring vital parameters (e.g., heart rate, blood pressure, body temperature, respiratory rate, electrocardiogram (ECG), etc.), safety functions (e.g., for detecting electrical voltages or currents, etc.), for which electrodes are required contacting the body or the skin.

An electrode on a smart clothing must, of course, also be electrically contacted in order to be electrically connected to the electrical system or electronics on the smart clothing. However, conventional connection techniques such as soldered or crimped joints are unsuitable in the textile industry. The methods are completely new for the textile industry and the corresponding process knowledge is lacking. In addition, connections produced in this manner are not very durable in washing processes to which textiles are subjected, and the methods are relatively complicated to carry out. Apart from this, it is understandably unfavorable to form a soldering point on a textile material, since this can easily damage or visibly impair the textile material. Owing to the size of such connections on smart clothing, crimped joints are also somewhat unfavorable, both in terms of appearance and comfort while wearing. The use of electrically conductive liquid adhesives or compounds to produce electrical connections on smart clothing would, in principle, also be possible. For example, silicone could be applied to a textile in liquid form. However, it is extremely difficult to work with liquid adhesives or liquid compounds in sewing workshops. The risk of soiling the textiles with glue or other liquid compounds is high, especially due to improper handling. Apart from this, the connections produced by such methods are also not process-safe and durable enough for everyday textile use.

There is also the problem that materials that are well suited for electrodes, such as silicone, PTFE or other plastics materials, adhere poorly or not at all to many materials that could be used to connect the electrode to the smart clothing.

Attaching electrodes to a textile using a method suitable for the textile industry and thus simultaneously producing an electrical connection is therefore a difficult task.

An object of the present invention is therefore that of allowing an electrode to be arranged on a smart clothing in a simple and process-safe manner while simultaneously producing an electrical connection.

This object can be achieved using an electrode arrangement in which an electrode carrier is provided in the form of a flat material piece consisting of a textile or non-textile material, the electrode carrier being connected on one side to an electrically conductive electrode and an electrically conductive thermoadhesive being in contact with the opposite side of the electrode carrier, the electrode and the thermoadhesive being electrically conductively connected for an electrical connection. For an electrical connection, the electrode and the thermoadhesive are electrically conductively connected, preferably via the electrode carrier or by direct contacting of the electrode material with the thermoadhesive or by providing electrical connections. Such an electrode arrangement can be prefabricated in the required shape and size and can be applied to a smart clothing in a simple and process-safe manner. The design of the electrode arrangement also makes it possible to use materials that are inherently incompatible with regard to adhesion. For example, in this manner a particularly suitable silicone electrode can be connected to the smart clothing using a thermoadhesive, even though silicone would not be suitable for being adhered using a thermoadhesive. This ensures that the requirements for the durability and process-safety of the connection and also the requirements for good electrical contacting can be met.

Electrodes from such an electrode arrangement can be produced very inexpensively in large sheets to form many units, it being particularly advantageously possible for the plastics areas forming the electrode to be applied by means of a printing process or extrusion. The thermoadhesive can be applied over the entire surface. Parts can then be punched out in the required shape and size and used as textile components. The application of such textile components to a smart clothing is also extremely simple under pressure and temperature, for example using a thermal transfer press.

In a preferred embodiment, the electrode carrier of the electrode arrangement is made of a textile material and the textile material of the electrode carrier is at least partially penetrated by the material of the electrode, preferably an electrically conductive plastics material. The electrical connection can thus be produced in a simple manner or the production of an electrical connection can at least be assisted. Producing an electrical connection is in particular easily possible if the textile material of the electrode carrier is also at least partially penetrated by the thermoadhesive.

If the electrode carrier is designed to be electrically conductive, the quality and durability of the electrical connection can be improved.

The present invention is described in more detail in the following with reference to the drawings which, by way of example, show schematic and non-limiting advantageous embodiments of the invention. In the drawings:

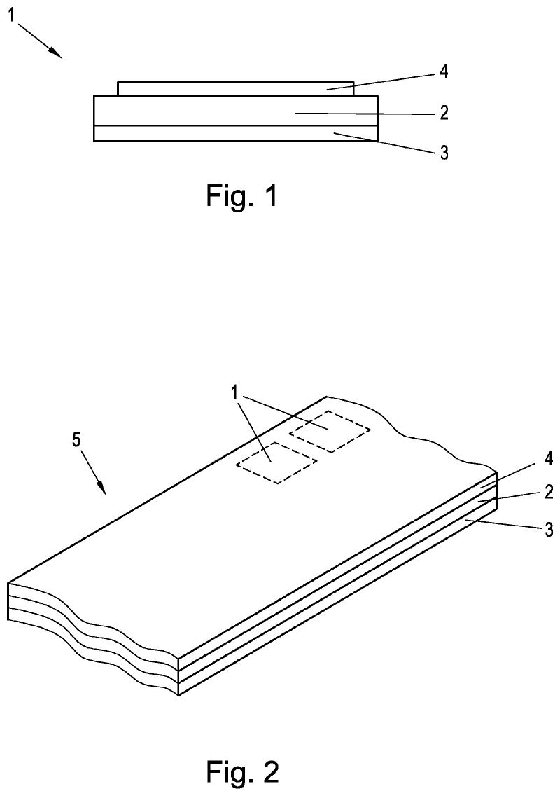

FIG. 1 shows an electrode arrangement,

FIG. 2 shows an electrode web for producing an electrode arrangement,

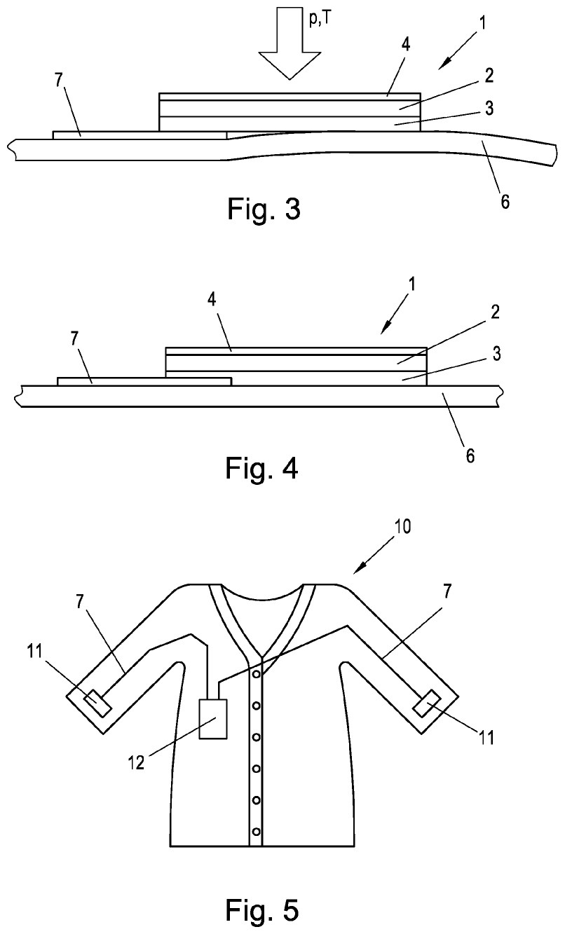

FIGS. 3 and 4 show the use of the electrode arrangement on a carrier material and

FIG. 5 shows a smart clothing together with an electrode from an electrode arrangement.

Textiles (textile materials) in the context of the invention comprise textile raw materials or non-textile raw materials which have been processed to form flat or three-dimensional textile structures by various methods. Textiles made of textile raw materials can also contain non-textile raw materials, and vice versa. Textile raw materials can include natural fibers (of plant or animal origin) or synthetic fibers (made of plastics materials), such as cotton, wool, polyester, or polyamide. Non-textile raw materials can e.g., also be plastics materials, leather, feathers, scales or metals. Typical flat structures are woven fabrics, knitwear, stitch fabrics, knitted fabrics, braided fabrics, stitch-bonded fabrics, nonwovens or felts, which are mostly in the form of fabric webs. Three-dimensional structures can be, for example, tubular structures, again in the form of woven fabrics, knitwear, stitch fabrics, knitted fabrics, braided fabrics, stitch-bonded fabrics, nonwovens or felts. A textile material can be sufficiently flexible in order to be processed to produce a clothing or to be able to be used in a clothing. A non-textile material may be a flat piece of a non-textile raw material, which may also have a certain amount of flexibility.

An electrode arrangement 1 according to the invention consists of a textile or a non-textile electrode carrier (patch or a small material piece) 2, on which an electrically conductive electrode 4 is applied on one side. The electrode carrier 2 is a material piece made of a textile or non-textile material which is sufficiently large and flat to be able to attach the required electrode 4 thereon. The electrode carrier 2 can be made from a textile material, such as a woven metal fabric, metal stitch fabric, knitted metal fabric or the like, or from a non-textile material such as a piece of leather, plastics material or metal. The electrode carrier 2 is advantageously the same size as or larger than the electrode 4.

Electrically conductive silicone, electrically conductive PTFE or a different electrically conductive plastics material, or a different electrically conductive material such as metal can be considered for the material for electrode 4. A plastics material can be made electrically conductive, for example, by adding electrically conductive fillers, such as aluminum, copper, graphite or carbon black. Electrically self-conducting polymers are likewise known, which can also be used. A material piece having an electrically conductive coating can also be considered for the electrode 4.

The electrode 4 can be applied to the electrode carrier 2 in a liquid manner, for example using a printing process, extruded onto the electrode carrier 2 (in the case of a plastics material), or adhered thereto using a suitable adhesive. In the case of an adhesive, an adhesive that ensures sufficient adhesion between the electrode 4 and the electrode carrier 2 is, of course, used. In order to ensure electrical contacting, the adhesive is also electrically conductive or electrical connections are to be provided through the adhesive layer. The electrode material or the adhesive can also at least partially penetrate the textile material of the electrode carrier 2 before hardening, which can assist the electrical contacting.

An electrically conductive thermoadhesive 3 is provided on the opposite side of the electrode arrangement 1.

A thermoadhesive 3 is an adhesive, usually in the form of a thermoplastic, which at normal use temperatures of a smart clothing, typically between −20° C. and 60° C., has a solid or resilient solid state and is usually not sticky (i.e., has no adhesive properties). Starting from a particular melting temperature (reaction temperature), typically between 125° C. and 250° C. (i.e., far above the use temperature), the thermoadhesive softens until it is flowable and also develops adhesive properties (thus becomes sticky). During cooling, the thermoadhesive hardens again and solidifies again, and the thermoadhesive loses its tackiness. Adhesive properties can also begin to develop below the melting temperature. When the thermoadhesive has melted, two adhesive partners that are to be connected to one another by the thermoadhesive are pressed together by pressure, such that the thermoadhesive connects to the two adhesive partners. After the thermoadhesive has hardened, a secure and permanent connection, which is also resilient according to the circumstances, is formed between the two adhesive partners.

The thermoadhesive 3 can be applied to the electrode carrier 2, for example applied in paste form, glued on or extruded on, or can be in the form of a net, woven fabric, or film or the like, and be placed loosely on the electrode carrier 2. To produce an electrical connection, the thermoadhesive 3 must also be electrically conductive.

In order to make such a thermoadhesive 3 electrically conductive, an electrically conductive material, for example graphite powder or similar conductive substances or fillers, can be added to the thermoadhesive 3. For example, a commercially available thermal transfer material can be in the form of granules for an extruder. When extruding the thermal transfer material to form a film or a fiber (e.g., as a raw textile material for the production of a textile material) or when extruding it onto the electrode carrier 2, the electrically conductive material, which can be in powder, paste or granular form, can be mixed in to make the extruded thermoadhesive electrically conductive. A textile material, e.g., a net or a woven fabric, made of thermoadhesive can then be produced from a fiber produced in this manner. A thermal transfer material in powder form can, e.g., be mixed with the electrically conductive material in order to produce a paste of thermoadhesive therefrom. A thermal transfer material in paste form can also be mixed with an electrically conductive material in powder or paste form in order to make the thermal transfer material electrically conductive. Examples of brands and manufacturers of thermal transfer materials include, inter alia, PLATAMID (from Arkema), DYNACOLL S (from Evonik) or TUBILUX TD 90 NF (from CHT). When a suitable material is selected for the thermal transfer material and the electrically conductive material, electrical connections having resistances of less than 10 ohms, preferably in the milliohm range, are obtained using a thermoadhesive produced from said material. Depending on the application, however, resistances in the kilo or megaohm range can also be sufficient.

In an advantageous embodiment, entire electrode webs 5 are produced in great length and width (as shown in FIG. 2) in that the electrode material is applied, preferably on the surface, on one side of a web of the electrode carrier 2, for example by extrusion, printing, gluing, etc., and in that the thermoadhesive 3 is applied to the other side, for example by extrusion, in the form of a paste, etc. When using a printing process for applying the electrode material, instead of a surface application, the electrode could also be printed in the required size and shape in order to save material. The electrode arrangements 1 can then be punched or cut out in the required size and shape from the electrode web 5 and processed further.

The electrode web 5 could also be produced without thermoadhesive 3. The thermoadhesive 3 can then be used as a separate adhesive patch (in the form of a textile or non-textile material such as a film, net, woven fabric, etc.). Alternatively, the thermoadhesive 3 could also be applied in paste form to the electrode arrangement 1 or to the carrier material 6, to which the electrode is to be attached, before use.

In a preferred embodiment of the electrode arrangement 1, an electrode carrier 2 made of a textile material is used, to which the electrode 4 was applied in liquid form or by extrusion, such that the electrically conductive electrode material at least partially penetrates the textile material of the electrode carrier 2. The electrically conductive thermoadhesive 3 is applied to the other side by extrusion. Ideally, the thermoadhesive 3 can already at least partially penetrate the textile material of the electrode carrier 2. In this manner, an electrical connection between the electrode 4 and the thermoadhesive 3 can already be produced during the production of the electrode arrangement 1. However, it can also be the case that only when the electrode arrangement 1 is applied, i.e., during pressing under pressure and temperature, does the electrically conductive thermoadhesive 3 melt to such an extent that the thermoadhesive 3 at least partially penetrates the textile material of the electrode carrier 2 in order to produce the electrical connection, or the penetration and thus the electrical contacting is improved.

In FIG. 3, the electrode arrangement 1 already includes the thermoadhesive 3, for example because this was applied to the electrode arrangement 1 together with the electrode arrangement 1 or subsequently. The electrode arrangement 1 is placed on a carrier material 6, for example a smart clothing. This is intended to produce an electrical connection between the electrode 4 of the electrode arrangement 1 and the carrier material 6 or an electrical conductor 7 on or in the carrier material 6. In addition, at least one electrical conductor 7, for example a wire, can therefore also be arranged between the electrode arrangement 1 and the carrier material 6 (as in FIG. 3). The electrical conductor 7 can rest on the carrier material 6 or be integrated in the carrier material 6. The electrical conductor 7 is, of course, stripped of its insulation at the end between the electrode arrangement 1 and the carrier material 6, in order to produce good electrical contacting. The carrier material 6 itself can also be locally electrically conductive, for example by incorporating electrically conductive fibers, for example made of metal, into the carrier material 6, which can also replace a conductor 7. The electrode arrangement 1 is then pressed under pressure p and temperature T (above the melting temperature of the thermal transfer material used) against the carrier material 6 (indicated by the arrow), for example using a thermal transfer press. Alternatively and equivalently, instead of an electrode arrangement 1 having thermoadhesive 3, a separate adhesive patch (as a net, woven fabric, film, etc.) made of thermoadhesive 3 could also be inserted between the electrode arrangement 1 and the carrier material 6. In this case, too, the thermoadhesive 3 melts during pressing under pressure p and temperature T and also connects to the carrier material 6 and to the electrode carrier 2 of the electrode arrangement 1 (as shown in FIG. 4) and at least partially surrounds any conductor 7 that may be present. After the thermoadhesive 3 has hardened, a secure, permanent electrical connection results between the electrode 4 and the conductor 7 and/or the carrier material 6. At the same time, the at least one conductor 7 can thereby be securely and permanently mechanically fixed on the carrier material 6.

The electrical connection through the electrode carrier 2 can be achieved by the electrode carrier 2 itself being electrically conductive, for example by suitable material selection, or by incorporating electrically conductive elements into the electrode carrier 2 which ensure the electrical connection, for example metal threads incorporated into a textile electrode carrier 2. The electrode carrier 2 can be designed, for example, as a woven wire cloth or knitted metal wire. However, the electrode carrier 2 can also be designed having an electrically conductive coating. If the electrode carrier 2 itself is electrically conductive, then the electrical connection is particularly good.

However, the electrical connection through the electrode carrier 2 can preferably also be achieved in that either the electrically conductive electrode material and/or the electrically conductive thermoadhesive 3 penetrates the material of the electrode carrier 2, for example by application or when the electrode arrangement 1 is applied by pressing under pressure p and temperature T, so that the electrode material and the thermoadhesive 3 contact. Therefore, the electrode carrier 2 is advantageously made of a textile material that can be easily penetrated by a liquid or melted electrode material and thermoadhesive 3.

Electrical and/or electronic parts, components or circuits can be integrated in the smart clothing 1 and can be electrically connected to the electrode 4 in this manner.

The use of an electrode carrier 2 is particularly advantageous if the thermoadhesive 3 would not adhere to the material of the electrode 4, as would be the case, for example, with an electrode 4 made of silicone or PTFE. However, since such an electrode material can be securely and reliably coated or applied as an electrode 4 on an electrode carrier 2 and the thermoadhesive 3 can also be securely connected to the electrode carrier 2, the electrode arrangement 1 according to the invention can be used to attach the electrode 4 to the carrier material 6 in a simple and process-safe manner.

In order to facilitate the application of the electrode arrangement 1 on the carrier material 6 of the smart clothing, an adhesive which is sticky at room temperature can also be provided on the side of the electrode arrangement 1 facing the carrier material 6, in order to temporarily fix the electrode arrangement 1 for the thermal transfer process. Such an adhesive is preferably only slightly adhesive and remains adhesive even after repeated placement of the electrode arrangement 1 on the carrier material 6, in order to be able to easily align the electrode arrangement 1 on the carrier material 6. Such an adhesive is preferably not provided over the entire surface of the electrode arrangement 1, but only partially and in a very small amount or grammage. Furthermore, such an adhesive preferably has a melting temperature which is below the melting temperature of the thermoadhesive 3 so as not to influence the thermal transfer process and the production of the electrical connection.

FIG. 5 shows an example of a smart clothing 10 (smart textile) having contact electrodes 11 on the cuffs in order to contact the skin of the person wearing it or another item in order to sense an electrical potential. The contact electrodes 11 have been applied to the smart clothing 10 in the form of electrode arrangements 1 using a thermal transfer process. The two contact electrodes 11 are connected, for example, to an evaluation unit 12 (e.g., a microprocessor having software or an electrical circuit) via conductors 7. The conductors 7 can be arranged on the carrier material 6 of the smart clothing 10 or integrated into the carrier material 6. In this example, an electrical voltage between the two contact electrodes 11 can be evaluated in the evaluation unit 12. The production of the smart clothing 10 can take place very easily and quickly with the use of the electrode arrangements 1.

1. Electrode arrangement comprising:
an electrically conductive electrode;
an electrically conductive thermoadhesive; and
an electrode carrier in the form of a flat material piece consisting of a textile or non-textile material, the electrode carrier being connected on one side to the electrically conductive electrode and the electrically conductive thermoadhesive being in contact with an opposite side of the electrode carrier, and in that the electrode and the thermoadhesive are electrically conductively connected to form an electrical connection.

2. The electrode arrangement according to claim 1, characterized in that the electrode carrier is made of a textile material and the textile material of the electrode carrier is at least partially penetrated by a material of the electrode.

3. The electrode arrangement according to claim 1, characterized in that the electrode carrier is made of a textile material and the textile material is at least partially penetrated by the thermoadhesive.

4. The electrode arrangement according to claim 1, characterized in that the electrode carrier is coated with the thermoadhesive.

5. The electrode arrangement according to claim 1, characterized in that the thermoadhesive is loosely in contact with the electrode carrier.

6. The electrode arrangement according to claim 1, characterized in that the electrode carrier is electrically conductive.

7. The electrode arrangement according to claim 6, characterized in that the electrode carrier has an electrically conductive coating.

8. The electrode arrangement according to claim 6, wherein the electrode carrier includes electrically conductive elements configured and arranged to form the electrical connection.

9. Smart clothing comprising;
a contact electrode;
a carrier material on which the contact electrode is arranged;
wherein the contact electrode includes an electrode arrangement according to claim 1, and the electrode arrangement is electrically connected to the smart clothing or a part thereof via the thermoadhesive.

10. The smart clothing according to claim 9, further including an electrical conductor which is at least partially surrounded by the thermoadhesive of the electrode arrangement.

11. The smart clothing according to claim 9, characterized in that the carrier material is at least partially electrically conductive and that the thermoadhesive at least partially contacts the electrically conductive carrier material.

12. Method for producing smart clothing with a contact electrode, the method including the following steps:
placing an electrode arrangement according to claim 1 on a carrier material of the smart clothing in order to form the contact electrode;
placing the thermoadhesive so as to face the carrier material; and
melting the thermoadhesive of the electrode arrangement under pressure and temperature, such that, after the thermoadhesive has hardened, the electrode arrangement is electrically connected to the carrier material.

13. The electrode arrangement of claim 2, wherein the textile material of the electrode carrier is at least partially penetrated by an electrically conductive plastic material.
